(19) United States
(12) Patent Application Publication
(10) Pub. No.: US 2012/0035155 A1
(43) Pub. Date: Feb. 9, 2012

(54) INDOLE-2-CARBOXAMIDE DEIVATIVE

(75) Inventors: Yuji Haga, Ibaraki (JP); Sayaka Mizutani, Ibaraki (JP); Nagaaki Sato, Saitama (JP)

(73) Assignee: MSD K.K., Chiyoda-ku, Tokyo (JP)

(21) Appl. No.: 13/263,801

(22) PCT Filed: Apr. 17, 2010
(86) PCT No.: PCT/JP2010/057848

§ 371 Date: Oct. 19, 2011

(30) Foreign Application Priority Data
Apr. 28, 2009 (JP) 2009-108823

(51) Int. Cl. A61K 31/4725; C07D 401/14; A61P 9/12; A61P 9/10; A61P 13/12; A61P 9/00; A61P 3/04; A61P 3/10; A61P 5/00; A61P 3/06; A61P 19/06; A61P 1/16; A61P 29/00; A61P 1/04; C07D 401/12

(52) U.S. Cl. 514/210.21; 546/146; 514/307

(57) ABSTRACT

To provide an indole compound having a human QRFP receptor antagonistic activity and useful as a preventive or a remedy for various disorders. A compound of a formula (I) or a pharmaceutically-acceptable salt thereof is provided: [wherein, R¹ represents a hydrogen atom, a halogen, a C₁₋₆ alkyl, etc,; R² represents a C₁₋₆ alkyl, or a halo-C₁₋₆ alkyl; R³ represents a hydrogen atom, a C₁₋₆ alkyl, etc.; R⁴ represents a hydrogen atom, a C₁₋₆ alkyl, a halo-C₁₋₆ alkyl, etc.; or R³ and R⁴, taken together with the nitrogen atom to which they bond, form a 3- to 6-membered aliphatic nitrogen-containing hetero ring; or R³ and Y³, taken together, form —CH₂—CH₂—; Y¹ and Y² are both hydrogen atoms; or Y¹ and Y², taken together, form —CH₂—CH₂—; and Y³ represents a hydrogen atom; or Y³ and R³, taken together, form —CH₂—CH₂—].

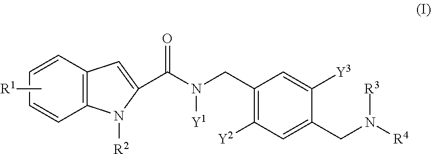

### TECHNICAL FIELD

The present invention relates to an indole-2-carboxamide derivative useful as medicines.

The compound acts as a human QRFP receptor (GPR103) antagonist and is useful as a preventive or remedy for obesity.

### BACKGROUND ART

QRFP43 is a peptide comprising 43 amino acids, and in 2003, this was reported to be an endogenous ligand of a QRFP43 receptor (GPR103) through bioinformatics and reverse pharmacological analysis, and later in 2006, this was first isolated from a rat brain (for example, see Non-Patent Reference 1). It is said that a QRFP43 analogue, for example, 26RFa binds to a QRFP43 receptor and exhibits the same activity as that of QRFP43 (for example, see Patent References 1 and 2). QRFP43 is much expressed in a central nervous system, especially in hypothalamus, and controls many functions in living bodies. Specifically, QRFP43 acts as an appetite stimulant in a central system, and remarkably promotes fat accumulation via secretion of various hormones or the action of a nervous system. It is known that continuous intracerebroventricular administration of QRFP43 induces obesity and insulin resistance based on those actions thereof. In addition, QRFP43 is related to secretion of hormones such as hypothalamo-hypophyseal hormone, etc.

QRFP43 or 26RFa expresses its function, when binding to a QRFP43 receptor (GPR103) existing in a central or peripheral nervous system. Accordingly, when QRFP43 or 26RFa is prevented from binding to a QRFP43 receptor (GPR103), then QRFP43 or 26RFa is prevented from expressing its function.

Compounds relating to the indole-2-carboxamide derivative of the invention are, for example, in WO2008/002674 (Patent Reference 3). In the compounds in this, a hydrogen atom bonds to the 1-position nitrogen atom of indole; on the other hand, in the compounds of the invention, an alkyl or a haloalkyl is in that site, thus the chemical structure differs in these points. In addition, the compounds described in Patent Reference 3 are a protein kinase inhibitor, and their use differs from those of the QRFP receptor (GPR103) antagonist of the invention.
[Non-Patent Reference 1] Proceedings of the National Academy of Sciences of the United States of America, Vol. 103, pp. 7438-7443, 2006.
[Patent Reference 1] WO01/16316
[Patent Reference 2] WO05/65702
[Patent Reference 3] WO2008/002674

### DISCLOSURE OF THE INVENTION

### Problems that the Invention is to Solve

When QRFP43 or 26RFa is prevented from binding to a QRFP 43 receptor (GPR103), then QRFP43 or 26RFa is prevented from expressing its function; and therefore, a substance antagonistic against the binding of QRFP43 to a QRFP 43 receptor (GPR103) is expected to be useful for prevention and treatment for various QRFP43 or 26RFa-related disorders, for example, circular system disorders such as hypertension, arteriosclerosis, renal diseases, heart diseases, vasospasm, etc.; for example, bulimia; metabolic disorders such as obesity, diabetes, hormone secretion abnormality, hypercholesterolemia, hyperlipidemia, gout, fatty liver, etc. The substance is useful to preventives or remedies for pain, circular rhythm disorders, for example, atherosclerosis, obesity-related gastroesophageal reflux, obesity hypoventilation syndrome (Pickwickian syndrome), hypertriglyceridemia, hypo-HDL cholesterolemia; circular system disorders such as coronary heart diseases (CRD), cerebrovascular disorders, stroke, peripheral vascular diseases, sudden death, etc.; pain, osteoporosis-related disorders, low back pain, anesthetic hypersensitivity, etc.

Accordingly, an object of the invention is to provide a QRFP43 or 26RFa antagonist useful as a preventive or a remedy for the above-mentioned disorders.

### Means for Solving the Problems

As a result of assiduous studies, the present inventors have found that an indole compound substituted with a specific benzylaminocarbonyl at the 2-position thereof has an excellent human QRFP receptor (GPR103) antagonistic activity, and have completed the present invention.

Specifically, the invention provides the following:

(1) A compound of a formula (1) or a pharmaceutically-acceptable salt thereof:

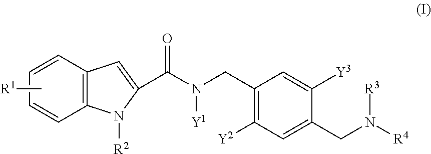

wherein,

R¹ represents a hydrogen atom, a halogen, a C₁₋₆ alkyl, a halo-C₁₋₆ alkyl, a C₁₋₆ alkyloxy, or a halo-C₁₋₆ alkyloxy;

R² represents a C₁₋₆ alkyl, or a halo-C₁₋₆ alkyl;

R³ represents a hydrogen atom, a C₁₋₆ alkyl, or a halo-C₁₋₆ alkyl;

R⁴ represents a hydrogen atom, a C₁₋₆ alkyl, a halo-C₁₋₆ alkyl, or a C₃₋₆ cycloalkyl; or R³ and R⁴, taken together with the nitrogen atom to which they bond, form a 3- to 6-membered aliphatic nitrogen-containing hetero ring;

or R³ and Y³, taken together, form —CH₂—CH₂—;

Y¹ and Y² are both hydrogen atoms; or Y¹ and Y², taken together, form —CH₂—CH₂—; and

Y³ represents a hydrogen atom; or Y³ and R³, taken together, form —CH₂—CH₂—,

(2) A pharmaceutical composition containing the compound of (1) or the pharmaceutically-acceptable salt thereof, and a pharmaceutically-acceptable carrier,

(3) A preventive or a remedy for obesity, comprising, as the active ingredient thereof, the compound of (1) or the pharmaceutically-acceptable salt thereof.

### BEST MODE FOR CARRYING OUT THE INVENTION

The invention is described in more detail hereinunder.

“Halogen” includes fluorine, chlorine, bromine and iodine.

“C₁₋₆ alkyl” includes a linear alkyl having from 1 to 6 carbon atoms or a branched alkyl having from 3 to 6 carbon atoms, concretely, for example, methyl, ethyl, n-propyl, isopropyl, n-butyl, isobutyl, sec-butyl, tert-butyl, n-pentyl, isopentyl, neopentyl, tert-amyl, 2-propyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, 1-ethyl-1-methylpropyl, etc.

“Halo-C₁₋₆ alkyl” includes a C₁₋₆ alkyl in which a part or all of the hydrogen atoms are substituted with a halogen, for example, fluoromethyl, difluoromethyl, trifluoromethyl, 2-fluoroethyl, 1,2-difluoroethyl, etc.

“C₁₋₆ alkyloxy” includes a group of an oxygen atom to which a C₁₋₆ alkyl bonds, concretely, methoxy, ethoxy, n-propyloxy, isopropyloxy, n-butyloxy, isobutyloxy, tert-butyloxy, n-pentyloxy, etc.

“Halo-C₁₋₆ alkyloxy” includes a group of an oxygen atom to which a halo-C₁₋₆ alkyl bonds, concretely, fluoromethoxy, chloromethoxy, difluoromethoxy, dichloromethoxy, trifluoromethoxy, trichloromethoxy, 2-fluoroethoxy, 1,2-difluoroethoxy, etc.

“C₃₋₈ cycloalkyl” includes a cycloalkyl having from 3 to 8 carbon atoms, for example, cyclopropyl, cyclobutyl, cyclopentyl, cyclohexyl, cycloheptyl, cyclooctyl, etc.

“Pharmaceutically-acceptable salts” of the derivatives of the formula (I) include ordinary salts acceptable as medicines. Their examples are acid-addition salts at the amine moiety of the compounds of the formula (I), or acid-addition salts at the nitrogen-containing hetero ring thereof; and base-addition salts at the acidic substituent, if any, of the compounds of the formula (I).

The acid-addition salts include inorganic acid salts such as hydrochlorides, sulfates, nitrates, phosphates, perchlorates, etc.; organic acid salts such as maleates, fumarates, tartrates, citrates, ascorbates, trifluoroacetates, etc.; sulfonates such as methanesulfonates, isothiocyanates, benzenesulfonates, p-toluenesulfonates, etc.

The base-addition salts include alkali metal salts such as sodium salts, potassium salts, etc.; alkaline earth metal salts such as calcium salts, magnesium salts, etc.; ammonium salts; organic amine salts such as trimethylamine salts, triethylamine salts, dicyclohexylamine salts, ethanolamine salts, diethanolamine salts, triethanolamine salts, procaine salts, N,N′-dibenzylethylenediamine salts, etc.

For illustrating the derivatives of the invention more concretely, the symbols used in the formula (I) are described in detail hereinunder with reference to their specific examples.

R¹ represents a hydrogen atom, a halogen, a C₁₋₆ alkyl, a halo-C₁₋₆ alkyl, a C₁₋₆ alkyloxy, or a halo-C₁₋₆ alkyloxy.

Concretely, R¹ includes a hydrogen atom; a halogen such as fluorine, chlorine, bromine, iodine; a C₁₋₆ alkyl such as methyl, ethyl, n-propyl, isopropyl, n-butyl, t-butyl; a halo-C₁₋₆ alkyl such as chloromethyl, trichloromethyl, fluoromethyl, trifluoromethyl, chloroethyl, fluoroethyl; a C₁₋₆ alkyloxy such as methoxy, ethoxy, n-propyloxy, isopropyloxy; a halo-C₁₋₆ alkyloxy such as chloromethoxy, trichloromethoxy, fluoromethoxy, trifluoromethoxy, fluoroethoxy, fluoropropyloxy; preferably a hydrogen atom or a halogen.

R² represents a C₁₋₆ alkyl, or a halo-C₁₋₆ alkyl.

Concretely, R² includes a C₁₋₆ alkyl such as methyl, ethyl, n-propyl, isopropyl, n-butyl, t-butyl; or a halo-C₁₋₆ alkyl such as chloromethyl, trichloromethyl, fluoromethyl, trifluoromethyl, chloroethyl, fluoroethyl; preferably a C₁₋₆ alkyl.

R³ represents a hydrogen atom, a C₁₋₆ alkyl, or a halo-C₁₋₆ alkyl;

R⁴ represents a hydrogen atom, a C₁₋₆ alkyl, a halo-C₁₋₆ alkyl, or a C₃₋₆ cycloalkyl; or R³ and R⁴, taken together with the nitrogen atom to which they bond, form a 3- to 6-membered aliphatic nitrogen-containing hetero ring;

or R³ and Y³, taken together, form —CH₂—CH₂—.

Concretely, R³ includes a hydrogen atom; a C₁₋₆ alkyl such as methyl, ethyl, n-propyl, isopropyl, n-butyl, t-butyl; a halo-C₁₋₆ alkyl such as chloromethyl, trichloromethyl, fluoromethyl, trifluoromethyl, chloroethyl, fluoroethyl; preferably a hydrogen atom or a C₁₋₆ alkyl.

Concretely, R⁴ includes a hydrogen atom; a C₁₋₆ alkyl such as methyl, ethyl, n-propyl, isopropyl, n-butyl, t-butyl; a halo-C₁₋₆ alkyl such as chloromethyl, trichloromethyl, fluoromethyl, trifluoromethyl, chloroethyl, fluoroethyl; a C₃₋₆ cycloalkyl such as cyclopropyl, cyclobutyl, cyclopentyl, cyclohexyl; preferably a hydrogen atom, a C₁₋₆ alkyl, or a C₃₋₆ cycloalkyl; more preferably methyl, cyclopentyl, cyclohexyl.

The 3- to 6-membered aliphatic nitrogen-containing hetero ring to be formed by R³ and R⁴ taken together includes aziridine, azetidine, pyrrolidine and piperidine, preferably azetidine and pyrrolidine, more preferably azetidine.

Preferred combinations of R³, R⁴ and Y³ include the following:

TABLE 1

| Y³ | R³ | R⁴ |
|---|---|---|
| H | H | C₁₋₆ alkyl |
| H | C₁₋₆ alkyl | C₁₋₆ alkyl |

| | |
|---|---|
| H | They form, taken together with the nitrogen |
| | atoms to which they bond, a 3- to 6-membered |
| | aliphatic nitrogen-containing hetero ring. |

| | |
|---|---|
| —CH₂—CH₂— | not specifically defined |
| —CH₂—CH₂— | C₁₋₆ alkyl |
| —CH₂—CH₂— | C₃₋₈ cycloalkyl |

Y¹ and Y² are both hydrogen atoms; or Y¹ and Y², taken together, form —CH₂—CH₂—;

Y³ represents a hydrogen atom; or Y³ and R³, taken together, form —CH₂—CH₂—.

Preferred embodiments of the compounds of the formula (I) are:

(1) the compounds of the formula (1), wherein

R¹ is a hydrogen atom or a halogen,

R² is a C₁₋₆ alkyl,

Y¹ and Y² are both hydrogen atoms,

Y³ and R³, taken together, form —CH₂—CH₂—,

R⁴ is a hydrogen atom, a C₁₋₆ alkyl or a C₃₋₈ cycloalkyl;

(2) the compounds of the formula (I), wherein

R¹ is a hydrogen atom or a halogen,

R² is a C₁₋₆ alkyl,

R³ is a hydrogen or a C₁₋₆ alkyl,

R⁴ is a C₁₋₆ alkyl or a C₃₋₈ cycloalkyl,

Y¹ and Y², taken together, form —CH₂—CH₂—,

Y³ is a hydrogen atom;

(3) the compounds of the formula (I), wherein

R¹ is a hydrogen atom or a halogen,

R² is a C₁₋₆ alkyl,

R³ and R⁴, taken together with the nitrogen atom to which they bond, form azetidine,

Y¹ and Y², taken together, form —CH₂—CH₂—;

Y³ is a hydrogen atom.

Concretely, examples of the compounds of the formula (I) include the following:
1-methyl-N-(1,2,3,4-tetrahydroisoquinolin-6-ylmethyl)-1H-indole-3-carboxamide,
N-[(2-ethyl-1,2,3,4-tetrahydroisoquinolin-6-yl)methyl]-1-methyl-1H-indole-2-carboxamide,
N-[(2-cyclopentyl-1,2,3,4-tetrahydroisoquinolin-6-yl)methyl]-1-methyl-1H-indole-2-carboxamide,
{6-[(dimethylamino)methyl]-3,4-dihydroisoquinolin-2(1H)-yl}(1-methyl-1H-indol-2-yl)methanone,
{6-[(methylamino)methyl]-3,4-dihydroisoquinolin-2(1H)-yl}(1-methyl-1H-indol-2-yl)methanone,
[6-{[cyclopropyl(methyl)amino]methyl}-3,4-dihydroisoquinolin-2(1H)-yl](1-methyl-1H-indol-2-yl)methanone,
[6-(azetidin-1-ylmethyl)-3,4-dihydroisoquinolin-2(1H)-yl](1-methyl-1H-indol-2-yl)methanone,
1-methyl-N-[(2-methyl-1,2,3,4-tetrahydroisoquinolin-6-yl)methyl]-1H-indole-2-carboxamide,
N-[(2-cyclobutyl-1,2,3,4-tetrahydroisoquinolin-6-yl)methyl]-1-methyl-1H-indole-2-carboxamide,
N-[(2-cyclohexyl-1,2,3,4-tetrahydroisoquinolin-6-yl)methyl]-1-methyl-1H-indole-2-carboxamide,
1-methyl-N-{[2-(tetrahydro-2H-pyran-4-yl)-1,2,3,4-tetrahydroisoquinolin-6-yl]methyl}-1H-indole-2-carboxamide,
(1-methyl-1H-indol-2-yl)[6-(pyrrolidin-1-ylmethyl)-3,4-dihydroisoquinolin-2(1H)-yl]methanone,
{6-[(ethylamino)methyl]-3,4-dihydroisoquinolin-2(1H)-yl}(1-methyl-1H-indol-2-yl)methanone,
[6-{{ethyl(methyl)amino]methyl}-3,4-dihydroisoquinolin-2(1H)-yl](1-methyl-1H-indol-2-yl)methanone,
(5-chloro-1-methyl-1H-indol-2-yl){6-[(dimethylamino)methyl]-3,4-dihydroisoquinolin-2(1H)-yl}methanone, and
(5-chloro-1-methyl-1H-indol-2-yl){6-[(methylamino)methyl]-3,4-dihydroisoquinolin-2(1H)-yl}methanone;
   preferably,
1-methyl-N-(1,2,3,4-tetrahydroisoquinolin-6-ylmethyl)-1H-indole-2-carboxamide,
N-[(2-ethyl-1,2,3,4-tetrahydroisoquinolin-6-yl)methyl]-1-methyl-1H-indole-2-carboxamide,
N-[(2-cyclopentyl-1,2,3,4-tetrahydroisoquinolin-6-yl)methyl]-1-methyl-1H-indole-2-carboxamide,
{6-[(dimethylamino)methyl]-3,4-dihydroisoquinolin-2(1H)-yl}(1-methyl-1H-indol-2-yl)methanone,
{6-[(methylamino)methyl]-3,4-dihydroisoquinolin-2(1H)-yl}(1-methyl-1H-indol-2-yl)methanone,
[6-{[cyclopropyl(methyl)amino]methyl}-3,4-dihydroisoquinolin-2(1H)-yl](1-methyl-1H-indol-2-yl)methanone, and
[6-(azetidin-1-ylmethyl)-3,4-dihydroisoquinolin-2(1H)-yl](1-methyl-1H-indol-2-yl)methanone.

### Production Methods for Compounds of Formula (I)

The compounds of the invention can be produced according to the methods mentioned below.

### Production Method 1:

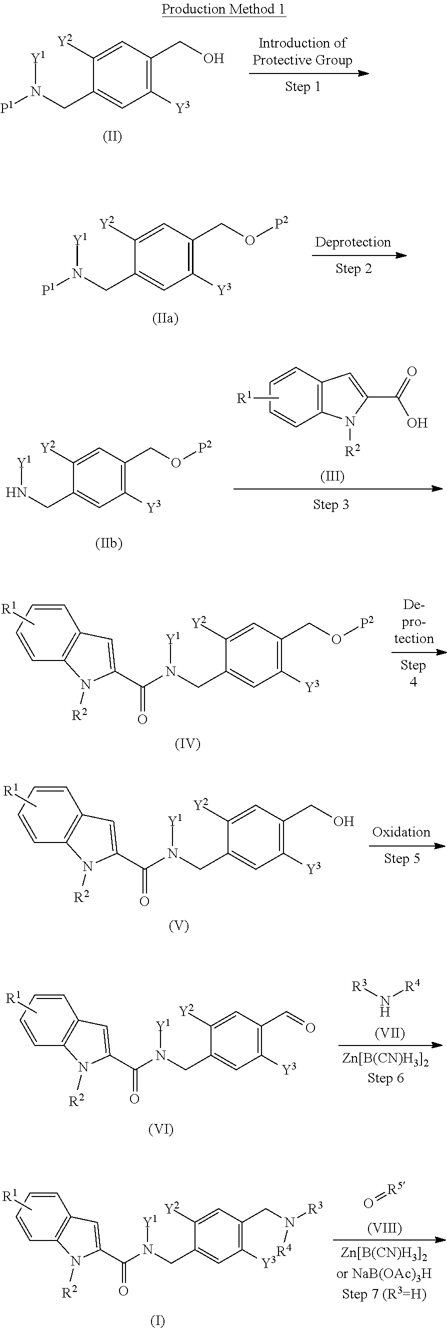

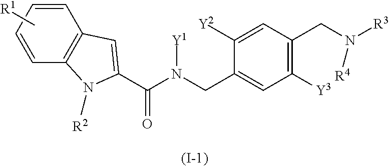

[In the formulae, P¹ represents an amino-protective group; P² represents a hydroxyl-protective group; R^{5′} represents a C₁₋₆ alkylene, a halo-C₁₋₆ alkylene, or a C₃₋₈ cycloalkylene; R⁵ represents a C₁₋₆ alkyl, a halo-C₁₋₆ alkyl, or a C₃₋₈ cycloalkyl; and the other symbols have the same meanings as above.]

### Step 1:

A hydroxyl-protective group (P²) is introduced into a compound of a formula (II) to give a compound of a formula (IIa). The protective group P² includes trimethylsilyl, t-butyldimethylsilyl, t-butyldiphenylsilyl, etc. The protective group introduction may be attained according to the methods described in Protective Groups in Organic Synthesis (T. W. Greene, John Wiley & Sons, 1981) or according to methods similar thereto.

For example, when a t-butyldiphenylsilyl group is introduced, the reaction may be attained in an organic solvent such as DMF, using t-butyldiphenylsilyl chloride and a base such as imidazole, at room temperature for 1 to 24 hours.

The compound of the formula (II) includes 1,1-dimethylethyl or 2-propenyl 3,4-dihydro-6-(hydroxymethyl)-2(1H)-isoquinolinecarboxylate.

### Step 2:

The amino-protective group (P¹) is removed from the compound of the formula (IIa) to give a compound of a formula (IIb). The protective group may be removed according to the methods described in “Protective Groups in Organic Synthesis” or according to methods similar thereto.

P¹ includes, for example, t-butyloxycarbonyl (Boc), benzyloxycarbonyl, p-methoxybenzyloxycarbonyl, allyloxycarbonyl, etc.; and preferred are t-butyloxycarbonyl and allyloxycarbonyl.

For example, Boc may be deprotected by treating the compound with trifluoroacetic acid (hereinafter referred to as “TFA”) at room temperature for 30 to 60 minutes.

### Step 3:

The compound of the formula (IIb) is reacted with a compound of a formula (III) through amidation in an organic solvent to give a compound of a formula (IV).

The amidation may be attained in any ordinary known manner, for example, according to a method of reacting the compound of the formula (IIb) and the compound of the formula (III) in the presence of a condensing agent, or a method comprising activating the carboxylic acid moiety of the compound of the formula (III) in an ordinary known manner to give a reactive derivative of the compound and reacting the derivative and the compound of the formula (IIb) through amidation (for these methods, referred to is “Basis and Experiments of Peptide Synthesis” (by Nobuo Izumiya, et al., Maruzen, 1983)).

For the reaction with a condensing agent, for example, the following method is mentioned.

Specifically, the compound of the formula (IIb) and the compound of the formula (III) are condensed in a reaction solvent with a condensing agent to give the compound of the formula (IV).

The amount of the compound of the formula (IIb) to be used may be from 1 to 3 mols per mol of the compound of the formula (III).

The condensing agent includes dicyclohexylcarbodiimide, 1-ethyl-3-(3-dimethylaminopropyl)-carbodiimide, O-(7-azabenzotriazol-1-yl)-1,1,3,3-tetramethyluronium hexafluorophosphate (hereinafter referred to as “HATU”); and its amount to be used may be from 1 to 3 mols per mol of the compound of the formula (III).

For promoting the reaction, hydroxybenzotriazole (hereinafter referred to as “HOBT”) may be added to the reaction system. The amount of HOBT to be used may be from 1 to 3 mols per mol of the compound of the formula (III).

The reaction solvent includes THF, 1,4-dioxane, N,N-dimethylformamide (hereinafter referred to as “DMF”), DMSO, dichloromethane, chloroform, pyridine and their mixed solvents.

The reaction temperature may be from 20 to 100° C., preferably from 20 to 50° C., and in general, the reaction may finish within 1 to 24 hours.

The compound of the formula (III) includes 1-methyl-1H-indole-2-carboxylic acid, 5-chloro-1-methyl-1H-indole-2-carboxylic acid, etc.

### Step 4:

The protective group P² is removed from the compound of formula (IV) to give a compound of a formula (V).

The protective group may be removed according to the methods described in Protective Groups in Organic Synthesis or according to methods similar thereto.

For example, when P² is a t-butyldiphenylsilyl group, the compound of the formula (IV) may be reacted with 1 equivalent amount of tetrabutylammonium fluoride in a solvent such as THF, at room temperature to 80° C.

### Step 5:

The compound of the formula (V) is oxidized in the presence of an oxidizing agent in an organic solvent to give a compound of a formula (VI).

The oxidizing agent may be any ordinary known one, including, for example, chromic acid, manganese dioxide, etc., preferably manganese dioxide.

The amount of the oxidizing agent to be used may be from 0.1 to 100 mols per mol of the compound of the formula (VI), preferably from 1 to 10 mols.

The organic solvent includes THF, dioxane, dimethyl ether, ethyl acetate, etc.

The reaction temperature may vary depending on the type of the oxidizing agent to be used. When manganese dioxide is used, the temperature may be from room temperature to 60° C., and in general, the reaction may finish within 1 to 10 hours.

### Step 6:

The compound of the formula (VI) is reacted with a compound of a formula (VII) through reductive alkylation in the presence of a reducing agent in an organic solvent to give the compound of the formula (I).

The amount of the compound of the formula (VII) to be used may be from 1 to 20 mols per mol of the compound of the formula (VI), preferably from 1 to 5 mols.

The reducing agent includes, for example, sodium cyanoborohydride, sodium triacetoxyborohydride, sodium borohydride, triethylsilane, etc. Of those, preferred are sodium cyanoborohydride and sodium triacetoxyborohydride.

The amount of the reducing agent to be used may be generally from 1 to 20 equivalents relative to 1 equivalent of the compound of the formula (VI), preferably from 1 to 5 equivalents.

Not specifically defined, the reaction solvent may be any one not interfering with the reaction, including, for example, methanol, ethanol, chloroform, methylene chloride, 1,2-dichloroethane, THF, 1,4-dioxane, etc.

The reaction may be attained in the presence of a base. The base includes, for example, triethylamine, triethylamine, N,N-diisopropylethylamine, N-methylmorpholine, N-methylpyrrolidine, N-methylpiperidine, etc.

The amount of the base to be used may be generally from 0 to 5 equivalents relative to 1 equivalent of the compound of the formula (VI), preferably from 0 to 2 equivalents.

Further, if desired, an additive such as zinc chloride, acetic acid or TFA may be added to the reaction system. For example, when zinc chloride is added, its amount to be added may be from 0.01 to 2 mols or so per mol of the reducing agent; and when acetic acid or TFA is added, its amount may be an excessive molar amount over the reducing agent.

The reaction temperature may be from 0 to 100° C., preferably from 0 to 50° C., and in general, the reaction may finish within 10 minutes to 48 hours, preferably within 10 minutes to 24 hours.

The compound of the formula (VII) includes methylamine, ethylamine, propylamine, cyclopropylamine, cyclobutylamine, cyclopentylamine, dimethylamine, ethylmethylamine, cyclopropylmethylamine, aziridine, azetidine, pyrrolidine, piperidine, etc.

### Step 7:

When R³ is H in the compound of the formula (I), the compound of the formula (I) may be reacted with a compound of a formula (VIII) according to the step 6 to give a compound of a formula (I-1). For the reaction condition, that in the step 6 is referred.

The compound of the formula (VIII) includes formaldehyde, acetaldehyde, cyclopropanone, etc.

### Production Method 2:

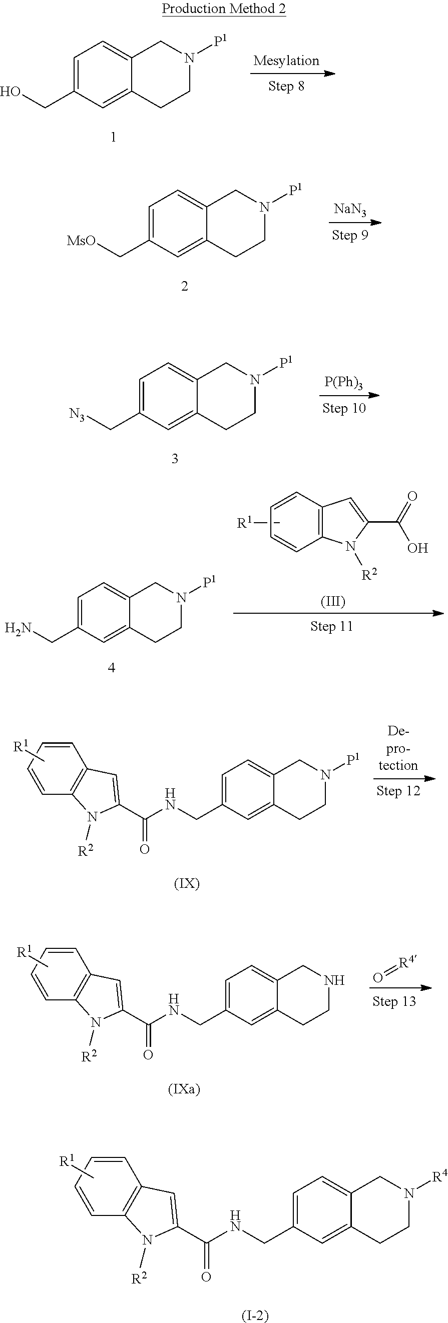

[In the formulae, R^{4′} represents a C₁₋₆ alkylene, a halo-C₁₋₆ alkylene or a C₃₋₈ cycloalkylene; and the other symbols have the same meanings as above.]

### Step 8:

An N-protected 6-tetrahydroisoquinoline-methanol (compound 1) is mesylated in an organic solvent in a known manner to give a compound 2.

The mesylation may be attained, for example, with methanesulfonyl chloride in the presence of an amine such as triethylamine in THF.

### Step 9:

The compound 2 is reacted with sodium azide in an organic solvent to give a compound 3.

The amount of sodium azide to be used may be from 1 to 50 mols per mol of the compound 2, preferably from 1 to 10 mols.

The organic solvent includes THF, N-methylpyrrolidone (hereinafter referred to as “NMP”), etc.

The reaction temperature may be from 0 to 200° C., preferably from room temperature to 100° C., and in general, the reaction may finish within 1 to 24 hours.

### Step 10:

The compound 3 is reduced in an organic solvent to give a compound 4.

Any method known by those skilled in the art is applicable to the reduction, including, for example, catalytic reduction with palladium-carbon, reduction with triphenyl phosphine, etc.

For example, in reduction with triphenyl phosphine, the amount of triphenyl phosphine to be used may be from 1 to 20 mols per mol of the compound 3, preferably from 1 to 5 mols.

The usable organic solvent includes THF, ether, etc.

The reaction temperature may be from room temperature to 100° C., preferably from room temperature to 80° C., and in general, the reaction may finish within 1 to 5 hours.

### Step 11:

The compound 4 is amidated with the compound of the formula (III) to give a compound of a formula (IX). The reaction condition is similar to that in the step 3.

### Step 12:

The compound of the formula (IX) is deprotected at the amino-protective group (P¹) thereof to give a compound of a formula (IXa). The method of deprotection is similar to the step 2.

### Step 13:

The compound of the formula (IXa) is reacted with a compound of a formula (VIIIa) through reductive alkylation to give a compound of a formula (I-2). The reaction may be attained in the same manner as that in the step 7.

In the above-mentioned production methods, when the reactants have amino, hydroxyl, carboxyl, oxo, carbonyl or the like not participating in the reaction, then the reaction in the production methods may be attained after the amino, hydroxyl, carboxyl, oxo or carbonyl is suitably protected with an amino-protective group, a hydroxy-protective group, a carboxyl-protective group or an oxo or carbonyl-protective group, and the protective group may be removed after the reaction.

Introduction and removal of the protective group may vary, depending on the type of the protective group and on the stability of the product compound. For example, according to the methods described in the above-mentioned reference, “Protective Groups in Organic Synthesis” or according to methods similar thereto, the protective group introduction and removal may be attained through solvolysis with an acid or a base, concretely, for example, according to a method of treating the protected compound with from 0.01 mol to a large excessive molar amount of an acid, preferably trifluoroacetic acid, formic acid, hydrochloric acid or the like, or with from an equimolar amount to a large excessive molar amount of a base, preferably potassium hydroxide, calcium hydroxide or the like; or through chemical reduction with a metal hydride complex or the like; or through catalytic reduction with a palladium-carbon catalyst, a Raney nickel catalyst or the like.

Not specifically defined, the amino-protective group may be any one having the intended function, for example, including an aralkyl such as benzyl, p-methoxybenzyl, trityl; a lower alkanoyl such as acetyl, pivaloyl; benzoyl; a lower alkyloxycarbonyl such as methoxycarbonyl, ethoxycarbonyl, tert-butoxycarbonyl; an alkyloxycarbonyl such as benzyloxycarbonyl; a lower alkylsilyl such as trimethylsilyl, tert-butyldimethylsilyl; tetrahydropyranyl; trimethylsilylethoxymethyl; a lower alkylsulfonyl such as methylsulfonyl, ethylsulfonyl; an arylsulfonyl such as benzenesulfonyl, toluenesulfonyl, etc. Especially preferred are acetyl, benzoyl, tert-butoxycarbonyl, trimethylsilylethoxymethyl, methylsulfonyl.

Not specifically defined, the hydroxy-protective group may be any one having the intended function, for example, including a lower alkyl such as methyl, ethyl, tert-butyl; a lower alkylsilyl such as trimethylsilyl, tert-butyldimethylsilyl; a lower alkyloxymethyl such as methoxymethyl, 2-methoxyethoxymethyl; tetrahydropyranyl; trimethylsilylethoxymethyl; an aralkyl such as benzyl, p-methoxybenzyl, 2,3-dimethoxybenzyl; an acyl such as acetyl, etc. Especially preferred are methyl, methoxymethyl, tetrahydropyranyl, trityl, trimethylsilylethoxymethyl, tert-butyldimethylsilyl, acetyl.

Not specifically defined, the carboxyl-protective group may be any one having the intended function, for example, including a lower alkyl such as methyl, ethyl, tert-butyl; a halo-lower alkyl such as 2,2,2-trichloroethyl; a lower alkenyl such as 2-propenyl; an aralkyl such as benzyl, p-methoxybenzyl, benzhydryl, trityl, etc. Especially preferred are methyl, ethyl, tert-butyl, 2-propenyl, benzyl, p-methoxybenzyl, benzhydryl.

Not specifically defined, the carbonyl-protective group may be any one having the intended function, for example, including an acetal and a ketal such as ethylene ketal, dimethyl ketal, S,S′-dimethyl ketal, etc.

Thus produced, the compounds of the formula (I), the compounds of the formula (I-1) and the compounds of the formula (I-2) may be readily isolated and purified in an ordinary separation method. The method includes, for example, solvent extraction, recrystallization, column chromatography, preparative thin-layer chromatography and the like.

### Pharmaceutical Composition Containing Compound of Formula (I)

The compounds of the formula (I) can be administered orally or parenterally, and after formulated into preparations suitable to such administration modes, the compounds are expected to be useful for prevention or treatment of circular system disorders such as hypertension, arteriosclerosis, renal diseases, heart diseases, vasospasm, etc.; for example, bulimia; metabolic disorders such as obesity, diabetes, hormone secretion abnormality, hypercholesterolemia, hyperlipidemia, gout, fatty liver, etc. The compounds are useful to preventives or remedies for pain, circular rhythm disorders, for example, atherosclerosis, obesity-related gastroesophageal reflux, obesity hypoventilation syndrome (Pickwickian syndrome), hypertriglyceridemia, hypo-HDL cholesterolemia; circular system disorders such as coronary heart diseases (CHD), cerebrovascular disorders, stroke, peripheral vascular diseases, sudden death, etc.; pain, osteoporosis-related disorders, low back pain, anesthetic hypersensitivity, etc.; and in particular, preventives or remedies for obesity.

In clinical use of the compounds of the invention, pharmaceutically-acceptable carriers may be added thereto, and after formulated into preparations suitable to their administration modes, the compounds can be administered. As the carriers, usable are various carriers generally used in the field of pharmaceutical preparations. Concretely, for example, they include gelatin, lactose, white sugar, titanium oxide, starch, crystalline cellulose, hydroxypropylmethyl cellulose, carboxymethyl cellulose, corn starch, microcrystalline wax, white vaseline, magnesium metasilicate aluminate, anhydrous calcium phosphate, citric acid, trisodium citrate, hydroxypropyl cellulose, sorbitol, sorbitan fatty acid ester, polysorbate, sucrose fatty acid ester, polyoxyethylene, hardened castor oil, polyvinylpyrrolidone, magnesium stearate, light silicic anhydride, talc, vegetable oil, benzyl alcohol, gum arabic, propylene glycol, polyalkylene glycol, cyclodextrin, hydroxypropylcyclodextrin, etc.

The preparations to be formulated as a mixture with the carrier include, for example, solid preparations such as tablets, capsules, granules, powders, suppositories; and liquid preparations such as syrups, elixirs, injections. These can be prepared according to ordinary methods in the filed of pharmaceutical preparations. The liquid preparations may be in such a form that is dissolved or suspended in water or in any other suitable medium before use. Especially for injections, the preparation may be dissolved or suspended, if desired, in a physiological saline or glucose solution, and a buffer and a preservative may be added thereto.

The preparations may contain the compound of the invention in an amount of from 1 to 99.9% by weight based on the pharmaceutical composition containing it, preferably from 1 to 60% by weight. The preparation may further contain any other compound effective for therapy.

Accordingly, the invention provides a pharmaceutical composition containing a pharmaceutically-acceptable carrier, and a therapeutically-effective amount of the compound or the pharmaceutically-acceptable salt thereof of the invention.

“Therapeutically-effective amount” as referred to herein means the amount of a drug capable of inducing biological or medical phenomena in tissues, systems, animals and humans.

Specifically, in case where the compound of the invention is used for prevention or treatment for the above-mentioned disorders, its dose and its administration frequency may vary depending on the sex, the age, the body weight and the condition level of the patient to which it is applied and on the type and the range of the intended therapeutical effect; and in general, in oral administration, the dose may be generally from 0.001 to 50 mg/kg (body weight)/day, and the preparation may be administered all at a time or may be administered in a few times as divided into a few portions. The dose is preferably from about 0.01 to about 25 mg/kg/day, more preferably from about 0.05 to about 10 mg/kg/day.

As combination therapy, the compound of the invention may be combined with a drug effective for hypertension, obesity-related hypertension, hypertension-related disorders, heart hypertrophy, left ventricular hypertrophy, metabolic disorders, obesity, obesity-related disorders and the like (hereinafter referred to as “co-drug”). These drugs may be administered simultaneously, separately or successively in prevention or treatment for the above-mentioned disorders.

In case where the compound of the invention is combined with one or more co-drugs, it may be a pharmaceutical composition for single administration. In combination therapy, however, the composition containing the compound of the invention and the co-drug may be packaged in different packages for one subject, and they may be administered simultaneously, separately or successively to the subject. As the case may be, they may be administered at different times.

For the dose of the co-drug, referred to is the ordinary dose in clinical use thereof; and the co-drug dose may be suitably selected depending on the subject to which it is administered, the administration route, the disorder of the subject, the combination mode of the co-drug, etc. The administration mode of the co-drug is not specifically defined, so far as the co-drug is combined with the compound of the invention in their clinical administration.

The administration mode includes, for example, 1) single administration of a single preparation containing both a compound of the invention and a co-drug; 2) simultaneous administration of two preparations through the same administration route, in which the two preparations are prepared separately and each separately contain a compound of the invention and a co-drug; 3) separate administration at different times of two preparations through the same administration route, in which the two preparations are prepared separately and each separately contain a compound of the invention and a co-drug; 4) simultaneous administration of two preparations through different administration routes, in which the two preparations are prepared separately and each separately contain a compound of the invention and a co-drug; 5) separate administration at different times of two preparations through different administration routes, in which the two preparations are prepared separately and each separately contain a compound of the invention and a co-drug (for example, administration of a compound of the invention followed by a co-drug, or administration in the reversed order). The dose ratio of the compound of the invention and the co-drug may be suitably determined depending on the subject to which they are administered, the administration route, the disorder of the subject, etc.

The co-drug for use in the invention includes, for example, remedies for diabetes, remedies for hyperlipidemia, remedies for hypertension, remedies for obesity, etc. Two or more different types of such co-drug may be combined in any desired ratio.

The usefulness of the compounds of the invention as medicines is proved, for example, by the following Pharmacological Test Example 1.

### Pharmacological Test Example 1

### QRFP43-Binding Inhibitory Test

A cDNA sequence [Accession No. NM_{—}198179] coding for a human QRFP receptor (GPR103) was cloned in an expression vector pEF1V5-H isB (by Invitrogen). The resulting expression vector was transfected in a host cell CHO-K1 NFAT β-Lactamase (by Aurora), according to a cationic lipid process [see Proceedings of the National Academy of Sciences of the United States of America, Vol. 84, p. 7413, 1987], thereby producing a QRFP receptor (GPR103).

A membrane sample prepared from the cells having expressed a QRFP receptor (GPR103) was incubated in an assay buffer (50 mM Tris-HCl, 1 mM EDTA and 0.1% BSA, pH 7.4) along with a test compound and 20,000 cpm [¹²⁵I] QRFP43 (by Perkin Elmer) therein, at 25° C. for 1 hour, and then filtered through a glass filter GF/C. After washed with 50 mM Tris-HCl (containing 2 mM EDTA, 10 mM MgCl₂ and 0.04% Tween-20) buffer (pH 7.4), the radioactivity on the glass filter was determined. The non-specific binding was determined in the presence of 1 μM peptide QRFP43, and the 50% inhibitory concentration (IC₅₀) of the test compound to the specific [¹²⁵I] QRFP43 binding was computed [see Endocrinology, Vol. 131, p. 2090, 1992]. The results are shown in Table.

TABLE 2

| Example | Structure | IC50(nM) |
|---|---|---|
| 1 | 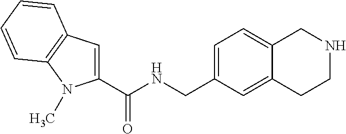 | 61 |
| 2 | 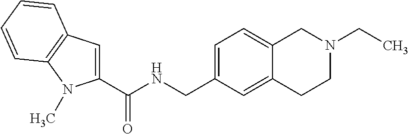 | 49 |
| 3 | 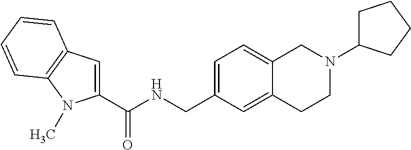 | 45 |
| 4 | 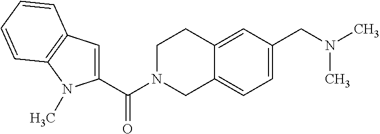 | 5.8 |
| 5 | 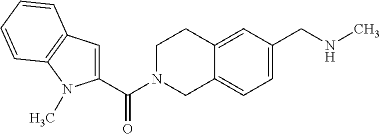 | 19 |
| 6 | 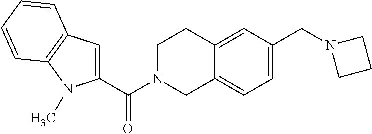 | 43 |
| 7 | 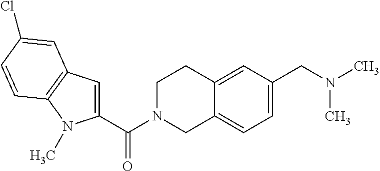 | 6.1 |
| 8 | 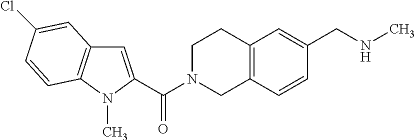 | 25 |

As in the above, the compounds of the invention strongly inhibited the binding of [¹²⁵I]QRFP43 to the QRFP receptor 43 (GPR103).

From the above results, the compounds of the invention are expected to be useful for prevention or treatment for various QRFP43 or 26RFa-related disorders, for example, circular system disorders such as hypertension, arteriosclerosis, renal diseases, heart diseases, vasospasm, etc.; for example, bulimia; and metabolic disorders such as obesity, diabetes, hormone secretion abnormality, hypercholesterolemia, hyperlipidemia, gout, fatty liver, etc. The substance is useful to preventives or remedies for pain, circular rhythm disorders, for example, atherosclerosis, obesity-related gastroesophageal reflux, obesity hypoventilation syndrome (Pickwickian syndrome), hypertriglyceridemia, hypo-HDL cholesterolemia; circular system disorders such as coronary heart diseases (CHD), cerebrovascular disorders, stroke, peripheral vascular diseases, sudden death, etc.; pain, osteoporosis-related disorders, low back pain, anesthetic hypersensitivity, etc.; especially to preventives or remedies for obesity.

### EXAMPLES

The invention is described more concretely with reference to Examples given below; however, the invention should not be limited to only these Examples. As column silica gel, used was Wakogel™ C-200 (Wako Pure Chemical Industries); as filled silica gel columns, used were disposable columns (Si series, NH series) (Moritex), cartridges for FLASH+™, KP-Sil or FPNH, FLASH12+M, FLASH25+S, FLASH25+M, FLASH40+M (Biotage Japan), TC-C18 (Agilent) or Extend-C18 (Zorbax); and for partitioning thin-layer chromatography, used as Kieselgel 60F254 (Merck). In mass spectrometry, used was Quarto II (Micromass). In ¹H NMR, used were JNM-AL400 (by JEOL) or MERCURYvx400 (by VARIAN) and ^{UNITY}INOV A400 (by VARIAN); and in mass spectrometry, used was ZQ2000 (by Waters).

### Example 1

### 1-Methyl-N-(1,2,3,4-tetrahydroisoquinolin-6-ylmethyl)-1H-indole-2-carboxamide

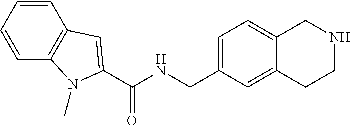

### (1) Production of 1,1-dimethylethyl 3,4-dihydro-6-methanesulfonyloxymethyl-2(1H)-isoquinolinecarboxylate

With cooling with ice, triethylamine (0.161 mL, 1.154 mmol) and methanesulfonyl chloride (0.054 mL, 0.693 mmol) were added to a tetrahydrofuran (5 mL) solution of 1,1-dimethylethyl 3,4-dihydro-6-(hydroxymethyl)-2(1H)-isoquinolinecarboxylate (152 mg, 0.577 mmol) with stirring, and the mixture was stirred with cooling with ice for 3.5 hours. The reaction liquid was diluted with ethyl acetate (20 mL), and washed with saturated sodium hydrogencarbonate solution (10 mL), saturated ammonium chloride solution and saturated brine. The organic layer was dried with magnesium sulfate, and the solvent was evaporated off under reduced pressure to give the entitled compound (231 mg, 100%) as a yellow oil.

### (2) Production of 1,1-dimethylethyl 6-azidomethyl-3,4-dihydro-2(1H)-isoquinolinecarboxylate

A mixture of 1,1-dimethylethyl 3,4-dihydro-6-methanesulfonyloxymethyl-2(1H)-isoquinolinecarboxylate (231 mg, 0.576 mmol), sodium azide (187 mg, 2.88 mmol) and N-methylpyrrolidone (2 mL) was stirred at room temperature for 1 hour. Water (20 mL) was added to the reaction liquid, and extracted with ethyl acetate (20 mL, twice). The organic layer was collected, washed with saturated brine, dried with magnesium sulfate, and the solvent was evaporated off under reduced pressure. The residue was purified through silica gel column chromatography to give the entitled compound (148 mg, 89%) as a colorless solid.

### (3) Production of 1,1-dimethylethyl 6-aminomethyl-3,4-dihydro-2(1H)-isoquinolinecarboxylate

A mixture of triphenyl phosphine (197 mg, 0.751 mmol), 1,1-dimethylethyl 6-azidomethyl-3,4-dihydro-2(1H)-isoquinolinecarboxylate (144 mg, 0.500 mmol), water (0.2 mL) and tetrahydrofuran (2 mL) was stirred under reflux for 2 hours. The reaction liquid was cooled to room temperature, then 1 N sodium hydroxide solution (5 mL) was added thereto, and extracted with chloroform (10 mL, three times). The organic layer was collected, dried with magnesium sulfate, and the solvent was evaporated off under reduced pressure. The residue was purified through silica gel column chromatography to give the entitled compound (131 mg, 100%) as a pale yellow solid.

### (4) Production of 1,1-dimethylethyl 6-({[(1-methyl-1H-indol-2-yl)carbonyl]amino}methyl)-3,4-dihydro-2(1H)-isoquinolinecarboxylate

O-(7-azabenzotriazol-1-yl)-1,1,3,3-tetramethyluronium hexafluorophosphate (207 mg, 0.545 mmol) was added to a mixture of 1,1-dimethylethyl 6-aminomethyl-3,4-dihydro-2(1H)-isoquinolinecarboxylate (130 mg, 0.496 mmol), 1-methyl-1H-indol-2-carboxylic acid (95 mg, 0.545 mmol), ethyldiisopropylamine (0.260 mL, 1.487 mmol) and N,N-dimethylformamide (1 mL) with stirring, and the mixture was stirred for 4 hours. 0.4; N sodium hydroxide solution (5 mL) was added to the reaction liquid, and stirred at room temperature for 2.5 days. The insoluble matter was collected through filtration, and washed with water. The resulting solid was dissolved in ethyl acetate, dried with magnesium sulfate, and the solvent was evaporated off under reduced pressure. The residue was purified through silica gel column chromatography to give the entitled compound (212 mg, 100%) as a yellow oil.

### (5) Production of 1-methyl-N-(1,2,3,4-tetrahydroisoquinolin-6-ylmethyl)-1H-indole-2-carboxamide

Trifluoroacetic acid (0.371 mL) was added to a chloroform (1 mL) solution of 1,1-dimethylethyl 6-({[(1-methyl-1H-indol-2-yl)carbonyl]amino}methyl)-3,4-dihydro-2(1H)-isoquinolinecarboxylate (206 mg, 0.482 mmol) with stirring, and the mixture was stirred for 2 hours. 1 N sodium hydroxide solution (10 mL) was added to the reaction liquid, and extracted with chloroform (10 mL, three times). The organic layer was collected, dried with magnesium sulfate, and the solvent was evaporated off under reduced pressure. The residue was crystallized from a mixed liquid of ethyl acetate and hexane to give the entitled compound (103 mg, 67%) as a pale yellow solid.

1H-NMR (400 MHz, CDCl₃) δ: 2.80 (t, J=6.0 Hz, 2H), 3.14 (t, J=6.0 Hz, 2H), 4.02 (s, 2H), 4.09 (s, 3H), 4.58 (d, J=5.6 Hz, 2H), 6.40-6.45 (m, 1H), 6.84 (d, J=0.7 Hz, 1H), 7.01 (t, J=6.0 Hz, 1H), 7.09-7.17 (m, 3H), 7.30-7.35 (m, 1H), 7.40 (dd, J=8.4, 0.9 Hz, 1H), 7.61 (dd, J=8.0, 1.0 Hz, 1H).

### Example 2

### N-(2-ethyl-1,2,3,4-tetrahydroisoquinolin-6-ylmethyl)-1-methyl-1H-indole-2-carboxamide,

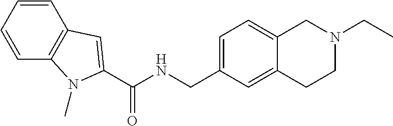

A methanol solution (1 mL, 0.3 mmol) of 0.3 M zinc bis(cyanotrihydroborate) prepared from sodium cyanotrihydroborate and zinc(II) chloride, and acetaldehyde (0.1 mL, 1.77 mmol) were added to a methanol (0.1 mL) suspension of 1-methyl-N-(1,2,3,4-tetrahydroisoquinolin-6-ylmethyl)-1H-indole-3-carboxamide (9.0 mg, 0.028 mmol), and stirred overnight at room temperature. 1 N sodium hydroxide solution (3 mL) was added to the reaction liquid, and extracted with chloroform (1 mL, three times). The organic layer was collected, and the solvent was evaporated off under reduced pressure. The residue was purified through silica gel column chromatography to give the entitled compound (4.3 mg, 44%) as a pale yellow solid.

1H-NMR (400 MHz, CDCl₃) δ: 1.22 (t, J=7.1 Hz, 3H), 2.65 (q, J=7.1 Hz, 2H), 2.80 (t, J=5.9 Hz, 2H), 2.95 (t, J=5.9 Hz, 2H), 3.69 (s, 2H), 4.09 (s, 3H), 4.58 (d, J=5.9 Hz, 2H), 6.45 (s, 1H), 6.84 (s, 1H), 7.04 (d, J=7.8 Hz, 1H), 7.10-7.17 (m, 3H), 7.30-7.34 (m, 1H), 7.39 (d, J=8.3 Hz, 1H), 7.61 (d, J=8.3 Hz, 1H).

### Example 3

### N-(2-cyclopentyl-1,2,3,4-tetrahydroisoquinolin-6-ylmethyl)-1-methyl-1H-indole-2-carboxamide

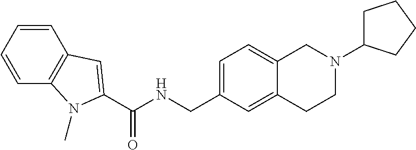

The entitled compound was produced in the same manner as in Example 2 but using cyclopentanone in place of acetaldehyde.

1H-NMR (400 MHz, CDCl₃) δ: 1.25 (m, 2H), 1.69-1.84 (m, 4H), 1.94-2.00 (m, 2H), 2.68-2.78 (m, 1H), 2.82 (t, J=5.9 Hz, 2H), 2.92 (t, J=5.9 Hz, 2H), 3.72 (s, 2H), 4.09 (s, 3H), 4.57 (d, J=5.9 Hz, 2H), 6.42 (s, 1H), 6.83 (s, 1H), 7.03 (d, J=7.3 Hz, 1H), 7.09-7.17 (m, 3H), 7.32 (m, 1H), 7.39 (dd, J=8.3, 1.0 Hz, 1H), 7.62 (d, J=7.8 Hz, 1H).

### Example 4

### [6-Dimethylaminomethyl-3,4-dihydro-2(1H)isoquinolin-2-yl](1-methyl-1H-indol-2-yl)methanone

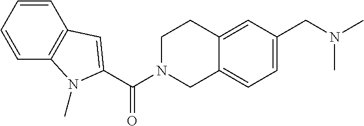

### (1) Production of 1,1-dimethylethyl 6-(t-butyldiphenylsilyloxymethyl)-3,4-dihydro-2(1H)-isoquinolinecarboxylate

T-butyldiphenylchlorosilane (557 mg, 2.03 mmol) and imidazole (288 mg, 4.22 mmol) were added in an N,N-dimethylformamide (6 mL) solution of 1,1-dimethylethyl 3,4-dihydro-6-(hydroxymethyl)-2(1H)-isoquinolinecarboxylate (445 mg, 1.69 mmol), and the mixture was stirred overnight at room temperature. The reaction liquid was diluted with ethyl acetate (20 mL), and washed with water and saturated brine. The organic layer was dried with magnesium sulfate, and the solvent was evaporated off under reduced pressure. The residue was purified through silica gel column chromatography to give the entitled compound (975 mg, 100%) as a colorless oil.

### (2) Production of 6-(t-butyldiphenylsilyloxymethyl)-1,2,3,4-tetrahydroisoquinoline

Trifluoroacetic acid (1.3 mL) was added to a chloroform (5 mL) solution of 1,1-dimethylethyl 6-(t-butyldiphenylsilyloxymethyl)-3,4-dihydro-2(1H)-isoquinolinecarboxylate (974 mg, 1.69 mmol), and the mixture was stirred for 3 hours. Chloroform (10 mL) was added to the reaction liquid, and washed with 1 N sodium hydroxide solution (10 mL) and 3 N sodium hydroxide solution (5 mL). The organic layer was dried with magnesium sulfate, and the solvent was evaporated off under reduced pressure. The residue was purified through silica gel column chromatography to give the entitled compound (644 mg, 95%) as a pale yellow solid.

### (3) Production of [6-(t-butyldiphenylsilyloxymethyl)-3,4-dihydro-2(1H)isoquinolin-2-yl](1-methyl-1H-indol-2-yl)methanone

The entitled compound was produced in the same manner as in Example 1(4) but using 6-(t-butyldiphenylsilyloxymethyl)-1,2,3,4-tetrahydroisoquinoline in place of 1,1-dimethylethyl 6-aminomethyl-3,4-dihydro-2(1H)-isoquinolinecarboxylate.

### (4) Production of [6-(hydroxymethyl)-3,4-dihydro-2(1H)isoquinolin-2-yl](1-methyl-1H-indol-2-yl)methanone

1 M tetrabutylammonium fluoride/tetrahydrofuran solution (2.52 mL) was added to a tetrahydrofuran (1 mL) solution of [6-(t-butyldiphenylsilyloxymethyl)-3,4-dihydro-2(1H)isoquinolin-2-yl](1-methyl-1H-indol-2-yl)methanone (703 mg, 1.26 mmol), and the mixture was stirred for 2 hours. Chloroform was added to the reaction liquid, and washed with saturated brine. The organic layer was dried with magnesium sulfate, and the solvent was evaporated off under reduced pressure. The residue was purified through silica gel column chromatography to give the entitled compound (388 mg, 96%) as a colorless solid.

### (4) Production of 2-[(1-methyl-1H-indol-2-yl)carbonyl]-1,2,3,4-tetrahydroisoquinoline-6-carbaldehyde

Manganese dioxide (1.05 mg, 12.1 mmol) was added to an ethyl acetate (24 mL) solution of [6-(hydroxymethyl)-3,4-dihydro-2(1H)isoquinolin-2-yl](1-methyl-1H-indol-2-yl)methanone (388 mg, 1.21 mmol), and the mixture was stirred for 6 hours. The insoluble matter was separated through filtration, and the solvent was evaporated off under reduced pressure. The residue was purified through silica gel column chromatography to give the entitled compound (362 mg, 94%) as a colorless solid.

### (5) Production of [6-dimethylaminomethyl-3,4-dihydro-2(1H)isoquinolin-2-yl](1-methyl-1H-indol-2-yl)methanone

The entitled compound was produced in the same manner as in Example 2 but using 2-[(1-methyl-1H-indol-2-yl)carbonyl]-1,2,3,4-tetrahydroisoquinoline-6-carbaldehyde and dimethylamine in place of acetaldehyde and 1-methyl-N-(1,2,3,4-tetrahydroisoquinolin-6-ylmethyl)-1H-indole-2-carboxamide.

1H-NMR (400 MHz, CDCl₃) δ: 2.25 (s, 6H), 2.94-2.99 (m, 2H), 3.40 (s, 2H), 3.85 (s, 3H), 3.96-4.00 (m, 2H), 4.91 (s, 2H), 6.69 (s, 1H), 7.12-7.19 (m, 4H), 7.29-7.33 (m, 1H), 7.38 (d, J=8.3 Hz, 1H), 7.65 (d, 0.1=7.8 Hz, 1H).

### Example 5

### [6-Methylaminomethyl-3,4-dihydro-2(1H)isoquinolin-2-yl](1-methyl-1H-indol-2-yl)methanone

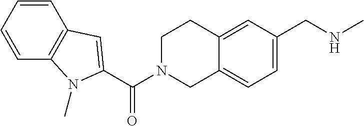

The entitled compound was produced in the same manner as in Example 2 but using 2-[(1-methyl-1H-indol-2-yl)carbonyl]-1,2,3,4-tetrahydroisoquinoline-6-carbaldehyde and methylamine in place of acetaldehyde and 1-methyl-N-(1,2,3,4-tetrahydroisoquinolin-6-ylmethyl)-1H-indole-2-carboxamide.

1H-NMR (400 MHz, CDCl₃) δ: 2.47 (s, 3H), 2.94-3.00 (m, 2H), 3.74 (s, 2H), 3.85 (s, 3H), 3.95-4.01 (m, 2H), 4.90 (s, 2H), 6.69 (s, 1H), 7.14-7.19 (m, 4H), 7.28-7.34 (m, 1H), 7.38 (d, J=7.8 Hz, 1H), 7.65 (d, J=8.0 Hz, 1H).

### Example 6

### [6-(Azetidin-1-ylmethyl)-3,4-dihydro-2(1H)isoquinolin-2-yl](1-methyl-1H-indol-2-yl)methanone

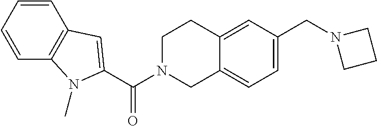

The entitled compound was produced in the same manner as in Example 2 but using 2-[(1-methyl-1H-indol-2-yl)carbonyl]-1,2,3,4-tetrahydroisoquinoline-6-carbaldehyde and azetidine in place of acetaldehyde and 1-methyl-N-(1,2,3,4-tetrahydroisoquinolin-6-ylmethyl)-1H-indole-2-carboxamide.

1H-NMR (400 MHz, CDCl₃) δ: 2.11 (p, J=7.1 Hz, 2H), 2.92-3.00 (m, 2H), 3.24 (t, J=7.1 Hz, 4H), 3.56 (s, 2H), 3.85 (s, 3H), 3.93-4.01 (m, 2H), 4.89 (s, 2H), 6.68 (s, 1H), 7.11-7.18 (m, 4H), 7.29-7.33 (m, 1H), 7.37 (d, J=7.8 Hz, 1H), 7.65 (d, J=8.0 Hz, 1H).

### Example 7

### [6-Dimethylaminomethyl-3,4-dihydro-2(1H)isoquinolin-2-yl](5-chloro-1-methyl-1H-indol-2-yl)methanone

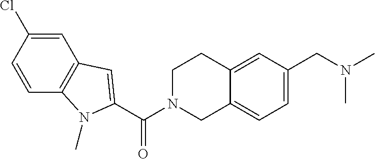

### (1) Production of 2-[(5-chloro-1-methyl-1H-indol-2-yl)carbonyl]-1,2,3,4-tetrahydroisoquinoline-6-carbaldehyde

The entitled compound was produced in the same manner as in Example 4, (3) and (4) but using 5-chloro-1-methyl-1H-indole-2-carboxylic acid in place of 1-methyl-1H-indole-2-carboxylic acid.

### (2) Production of [6-dimethylaminomethyl-3,4-dihydro-2(1H)isoquinolin-2-yl](5-chloro-1-methyl-1H-indol-2-yl)methanone

The entitled compound was produced in the same manner as in Example 2 but using 2-[(5-chloro-1-methyl-1H-indol-2-yl)carbonyl]-1,2,3,4-tetrahydro isoquinoline-6-carbaldehyde and dimethylamine in place of acetaldehyde and 1-methyl-N-(1,2,3,4-tetrahydroisoquinolin-6-ylmethyl)-1H-indole-2-carboxamide.

1H-NMR (400 MHz, DMSO-D₆) δ: 2.25 (s, 6H), 2.90 (t, J=5.6 Hz, 2H), 3.46-3.56 (m, 2H), 3.75 (s, 3H), 3.78-3.90 (m, 2H), 4.80 (s, 2H), 6.73 (s, 1H), 7.15 (s, 2H), 7.25 (dd, J=8.8, 2.2 Hz, 1H), 7.56 (d, J=9.0 Hz, 1H), 7.67 (d, J=1.7 Hz, 1H).

### Example 8

### [6-methylaminomethyl-3,4-dihydro-2(1H)isoquinolin-2-yl](5-chloro-1-methyl-1H-indol-2-yl)methanone

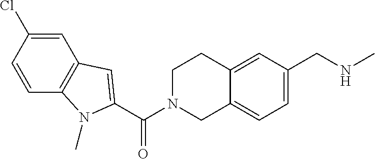

The entitled compound was produced in the same manner as in Example 2 but using 2-[(5-chloro-1-methyl-1H-indol-2-yl)carbonyl]-1,2,3,4-tetrahydro isoquinoline-6-carbaldehyde and methylamine in place of acetaldehyde and 1-methyl-N-(1,2,3,4-tetrahydroisoquinolin-6-ylmethyl)-1H-indole-2-carboxamide.

1H-NMR (400 MHz, DMSO-D₆) δ: 2.35 (s, 3H), 2.89 (t, J=5.5 Hz, 2H), 3.75-3.86 (m, 7H), 4.80 (s, 2H), 6.72 (s, 1H), 7.20-7.27 (m, 3H), 7.56 (d, J=8.8 Hz, 1H), 7.67 (d, J=1.7 Hz, 1H), 8.26 (s, 1H).

### INDUSTRIAL APPLICABILITY

The compounds of the invention are expected to be useful in prevention or treatment for various QRFP43 or 26RFa-related disorders, for example, circular system disorders such as hypertension, arteriosclerosis, renal diseases, heart diseases, vasospasm, etc.; for example, bulimia; metabolic disorders such as obesity, diabetes, hormone secretion abnormality, hypercholesterolemia, hyperlipidemia, gout, fatty liver, etc. Accordingly, the compounds are useful to preventives or remedies for pain, circular rhythm disorders, for example, atherosclerosis, obesity-related gastroesophageal reflux, obesity hypoventilation syndrome (Pickwickian syndrome), hypertriglyceridemia, hypo-HDL cholesterolemia; circular system disorders such as coronary heart diseases (CHD), cerebrovascular disorders, stroke, peripheral vascular diseases, sudden death, etc.; pain, osteoporosis-related disorders, low back pain, anesthetic hypersensitivity, etc.

1. A compound of a formula (I) or a pharmaceutically-acceptable salt thereof: 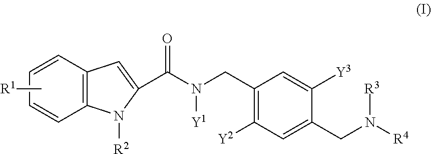
wherein,
R¹ represents a hydrogen atom, a halogen, a C₁₋₆ alkyl, a halo-C₁₋₆ alkyl, a C₁₋₆ alkyloxy or a halo-C₁₋₆ alkyloxy;
R² represents a C₁₋₆ alkyl, or a halo-C₁₋₆ alkyl;
R³ represents a hydrogen atom, a C₁₋₆ alkyl, or a halo-C₁₋₆ alkyl;
R⁴ represents a hydrogen atom, a C₁₋₆ alkyl, a halo-C₁₋₆ alkyl, or a C₃₋₆ cycloalkyl; or R³ and R⁴, taken together with the nitrogen atom to which they bond, form a 3- to 6-membered aliphatic nitrogen-containing hetero ring;
or R³ and Y³, taken together, form —CH₂—CH₂—;
Y¹ and Y² are both hydrogen atoms; or Y¹ and Y², taken together, form —CH₂—CH₂—; and
Y³ represents a hydrogen atom; or Y³ and R³, taken together, form —CH₂—CH₂—.

2. The compound or the pharmaceutically-acceptable salt thereof as claimed in claim 1, wherein R¹ is a hydrogen atom or a halogen.

3. The compound or the pharmaceutically-acceptable salt thereof as claimed in claim 1, wherein R² is a C₁₋₆ alkyl.

4. The compound or the pharmaceutically-acceptable salt thereof as claimed in claim 1, wherein R³ is a hydrogen atom or a C₁₋₆ alkyl.

5. The compound or the pharmaceutically-acceptable salt thereof as claimed in claim 1, wherein R³ and R⁴, taken together, form azetidine.

6. The compound or the pharmaceutically-acceptable salt thereof as claimed in claim 1, wherein R³ and Y³, taken together, form —CH₂—CH₂—.

7. The compound or the pharmaceutically-acceptable salt thereof as claimed in claim 1, wherein R⁴ is a hydrogen atom, a C₁₋₆ alkyl, or a C₃₋₆ cycloalkyl.

8. The compound or the pharmaceutically-acceptable salt thereof as claimed in claim 1, wherein Y¹ and Y² are both hydrogen atoms.

9. The compound or the pharmaceutically-acceptable thereof salt as claimed in claim 1, wherein Y¹ and Y², taken together, form —CH₂—CH₂—.

10. The compound or the pharmaceutically-acceptable salt thereof as claimed in claim 1, which is selected from a group consisting of:
methyl-N-(1,2,3,4-tetrahydroisoquinolin-6-ylmethyl)-1H-indole-2-carboxamide,
1-methyl-N-[(2-ethyl-1,2,3,4-tetrahydroisoquinolin-6-yl)methyl]-1H-indole-2-carboxamide,
N-[(2-cyclopentyl-1,2,3,4-tetrahydroisoquinolin-6-yl)methyl]-1-methyl-1H-indole-2-carboxamide,
{6-[(dimethylamino)methyl]-3,4-dihydroisoquinolin-2(1H)-yl}(1-methyl-1H-indol-2-yl)methanone,
{6-[(methylamino)methyl]-3,4-dihydroisoquinolin-2(1H)-yl}(1-methyl-1H-indol-2-yl)methanone,
[6-{[cyclopropyl(methyl)amino]methyl}-3,4-dihydroisoquinolin-2(1H)-yl] (1-methyl-1H-indol-2-yl)methanone, and
[6-(azetidin-1-ylmethyl)-3,4-dihydroisoquinolin-2(1H)-yl](1-methyl-1H-indol-2-yl)methanone.

11. A pharmaceutical composition containing the compound of claim 1, and a pharmaceutically-acceptable carrier.

12. (canceled)
